(19) United States
(12) Patent Application Publication
(10) Pub. No.: US 2014/0017298 A1
(43) Pub. Date: Jan. 16, 2014

(54) BIOPOLYMER HOOKS TO CREATE COATINGS ON LIPOSOMES

(71) Applicant: THE ADMINISTRATORS OF THE TULANE EDUCATIONAL FUND, (US)

(72) Inventors: Vijay JOHN, Destrehan, LA (US); Srinivasa RAGHAVAN, Silver Springs, MD (US); Rubo ZHENG, New Orleans, LA (US)

(73) Assignee: THE ADMINISTRATORS OF THE TULANE EDUCATIONAL FUND, New Orleans, LA (US)

(21) Appl. No.: 13/841,849

(22) Filed: Mar. 15, 2013

Related U.S. Application Data
(60) Provisional application No. 61/618,497, filed on Mar. 30, 2012.
(60) Provisional application No. 61/653,767, filed on May 31, 2012.

(51) Int. Cl. A61K 9/127

(52) U.S. Cl. CPC A61K 9/1271
USPC 424/450; 514/44 R; 514/1.1

(57) ABSTRACT

The disclosed invention involves creating coatings on liposomes to increase stability within the body for drug delivery. The present invention includes a composition used for drug delivery, comprising liposomes and hydrophobically modified polysaccharides with alkyl groups, wherein the alkyl groups physically attach to and coat the liposomes.

### CROSS-REFERENCES TO RELATED APPLICATIONS

This is a non-provisional patent application of U.S. Provisional Patent Application Ser. No. 61/618,497, filed 30 Mar. 2012; and of U.S. Provisional Patent Application Ser. No. 61/653,767, filed 31 May 2012.

Priority of U.S. Provisional Patent Application Ser. No. 61/618,497, filed 30 Mar. 2012; and U.S. Provisional Patent Application Ser. No. 61/653,767, filed 31 May 2012, each of which is hereby incorporated herein by reference, is hereby claimed.

Incorporation herein by reference are U.S. patent application Ser. No. 13/559,471, filed 26 Jul. 2012; and U.S. Provisional Patent Application Ser. No. 61/572,992, filed 26 Jul. 2011. Also incorporated herein by reference are U.S. patent application Ser. No. 13/502,047, filed 13 Apr. 2012 (published as US Patent Application Publication No. US2013/0058724); International Application Number PCT/US2010/052713, filed 14 Oct. 2010 (published as International Application Publication No. WO 2011/047181); and U.S. Provisional Patent Application Ser. No. 61/251,632, filed 14 Oct. 2009. Also incorporated herein by reference is U.S. patent application Ser. No. 13/291,038, filed 7 Nov. 2011; and U.S. Provisional Patent Application Ser. No. 61/456,358, filed 5 Nov. 2010. Also incorporated herein by reference is U.S. patent application Ser. No. 12/420,655, filed 8 Apr. 2009; and U.S. Provisional Patent Application Ser. No. 61/123,413.

### STATEMENT REGARDING FEDERALLY SPONSORED RESEARCH OR DEVELOPMENT

Funding was received from the US Department of Defense, Grant: W81XWH-10-1-0377. The United States government has certain rights in this invention.

### COMPACT DISK SUBMISSION

Not Applicable.

### BACKGROUND OF THE INVENTION

1. Field of the Invention

The present invention relates to liposomes, and specifically liposomes that encapsulate active molecules for drug delivery. More particularly, the present invention relates to coating liposomes with a hydrophobically modified polysaccharide and preferably used for delivery of compounds in a human body.

2. General Background of the Invention

### Liposomes are:

Composed of phospholipids and are mimics for cell membranes.
Encapsulate active molecules for drug delivery.
Liable to be destroyed by pH, bile salts, and pancreatic lipase in the gastrointestinal tract.
Rapidly removed by Kupffer cells in the liver and fixed macrophages in the spleen.

### Liposomes Decorated by Polyethylene Glycol (PEG):

Improve structural stability;
Improve drug delivery efficiency:

a. limit binding of serum opsonins;

b. avoid uptake by the reticuloendothelial system (RES) and extends circulation half-life in vivo.

PEG-liposomes are PEG derivatized phospholipids mixed with native phospholipids to prepare PEG-liposomes.

### Chitosan is:

Produced commercially from crustaceans such as crabs and shrimp.
A copolymer of N-acetylglucosamine and glucosamine.
Biocompatible, biodegradable. It has properties of mucosal adhesion, bioactivity and bioresorption.

Chitosan is a common biocompatible and biodegradable polymer. It is a derivative of chitin, which is obtained from seafood-processing wastes (crab, shrimp and lobster shells). The production of chitosan is thereby environmentally friendly (C. M. Aberg, et al.), and the polymer is considered fully biocompatible with significant applications in drug delivery and hemostasis (J. Yang, et al.; Q. Z. Wang, et al.; B. C. Dash, et al.; A. El-Mekawy, et al.). Scientifically, it is a linear copolymer composed of glucosamine and N-acetylglucosamine residues. Importantly, this polycationic biopolymer is easily obtained by alkaline deacetylation of chitin, which is the main component of the exoskeleton of crustaceans, such as shrimp, and due to these favorable properties, the interest in chitosan and its derivatives has been increased in recent years.

### Hydrophobically Modified Chitosan (HMC):

Hydrophobically-modified chitosan (hm-chitosan or hmC or HMC) can be synthesized by attaching alkyl tails to some of the amine moieties on the chitosan backbone. Long-chain aldehydes can be grafted to the chitosan backbone using reductive amination. Hydrophobically modify chitosan can interact with liposomes. Hydrophobically modified water soluble polymers can be anchored to the vesicle membrane by inserting the hydrophobic groups into vesicles bilayers. A system-spanning 3D polymer network forms. Implication of water based gel formation where the liposomes are the nodes in the gel network. See Jae-Ho Lee, Srinivasa R. Raghavan, et al. Langmuir 2005, 21, 26-33; Gregory F. Payne and Srinivasa R.raghavan, Soft Matter 2007, 3, 521; Jae-Ho Lee, Srinivasa R. Raghavan, et al. Physical Review Letters 2006, 96, 048102.

Attached to U.S. Provisional Patent Application No. 61/618,497 is an 18-page paper entitled “Biopolymer ‘hooks’ to Create Coatings on Liposomes” which is hereby incorporated herein by reference.

Incorporated herein by reference is U.S. patent application Ser. No. 12/420,655, filed Apr. 8, 2009. It is possible to coat the tubular liposomes mentioned herein with a hydrophobically modified polysaccharide (such as chitosan, carboxymethyl cellulose, hydroxypropyl cellulose, alginate, guar, starch, dextran, poly lactate, poly ascorbate, gelatin, xantham gum, glycans, welan guam, gellan gum, diutan gum, pullulan, and arabinoxylans and mixtures thereof) by the same methods described in U.S. patent application Ser. No. 12/420,655.

### BRIEF SUMMARY OF THE INVENTION

In previous research, it has been shown that liposomes can be combined with hydrophobically modified chitosan (HMC) to produce a gel. In the present invention, diluting the solution by decreasing the amount of a hydrophobically modified polysaccharide (such as chitosan, carboxymethyl cellulose, hydroxypropyl cellulose, alginate, guar, starch, dextran, poly lactate, poly ascorbate, gelatin, xantham gum, glycans, welan guam, gellan gum, diutan gum, pullulan, and arabinoxylans and mixtures thereof) and decreasing the number of liposomes results in the hydrophobically modified polysaccharide creating a coating on the liposomes, instead of a gel network as seen in prior research. Dilution during preparation results in the liposomes being further apart so that the HMC does not bridge the liposomes. At each concentration of liposomes, there will be some high concentration of HMC that will gel the liposomes. Washing the liposome solutions will assist with getting rid of excess HMC that is not attached to the liposomes. The coating of hydrophobically modified polysaccharide is a protective coating that extends circulation life of the liposomes in the body and allows for slower diffusion of the drugs from the liposome into the body. The coating described in the present invention is similar to putting a coating on aspirin (enteric coating).

Hypothesis—Can we coat liposomes with a hydrophobically modified polysaccharide (such as chitosan, carboxymethyl cellulose, hydroxypropyl cellulose, alginate, guar, starch, dextran, poly lactate, poly ascorbate, gelatin, xantham gum, glycans, welan guam, gellan gum, diutan gum, pullulan, and arabinoxylans and mixtures thereof), at appropriate concentrations where the polymer interacts with a single liposome? We believe it is possible to add dilute low molecular weight HMC (LHMC) (or another hydrophobically modified polysaccharide) solutions to liposome suspensions to minimize interaction with multiple liposomes. The molecular weight is approximately 50 k-190 k Daltons, preferably 50 k Daltons to produce a coating on the liposomes. A molecular weight closer to 190 k Daltons will probably bridge the liposomes and form gels.

Liposome Preparation

Lipids: Dipalmitoylphosphatidylcholine (DPPC) Dimyristoyl-sn-Glycero-3-PhosphoGlycerol (DMPG), or any lipid available at http://avantilipids.com; Method: Lipid film hydration.

Incubating liposomes in LHMC solutions—LHMC solution added to the liposome suspension and homogenized by gently stirring. The suspension is then incubated for 30 min at room temperature.

Understanding Interaction Between Liposomes and LHMC from Viscosity (See FIG. 6):

At lower concentrations, coating liposomes with LHMC reduces the entanglement of the polymer chains and thus reduces the viscosity of the polymer solutions. At higher concentrations, liposomes may act as connection nodes for LHMC chains therefore increasing LHMC solution viscosity. At lower concentrations of LHMC and liposome, coating liposomes with LHMC reduces the entanglement of the polymer chains and thus reduces solution viscosity. At higher concentration of LHMC and liposome, liposome may act as connection node for LHMC chains thereby increasing LHMC solution viscosity.

Cryo-TEM (Cryogenic Transmission Electron Microscopy) characterization: coating liposomes with LHMC (or another hydrophobically modified polysaccharide)—A dark layer around liposomes is observed after incubating liposomes in LHMC solution. The mass ratio of LHMC to lipid is 0.1-1.0, preferably 0.4 (see FIG. 3). The thickness of the coating layer increases as the mass ratio of polymer to lipid increases (see FIG. 4).

Liposome-LHMC transitions to gel (see FIG. 1a)—the addition of liposomes to LHMC solution results in a gel. Dye used in samples: 0.0005 wt % methylene blue. Understanding gel network structure by Cryo SEM (see FIG. 2c)—liposomes act as nodes to connect polymer chains.

Understanding gelation through dynamic rheology: The addition of liposomes to the polymer solution results in gel formation (see FIG. 5). Jae-Ho Lee, Srinivasa R. Raghavan, et al. Langmuir 2005, 21, 26-33; K. Almdal, J. Dyre, S. Hvidt, O. Kramer, Polymer Gels and Networks 1993, 1, 5-17.

Adding low concentrations of liposomes to dilute LHMC (or other dilute hydrophobically modified polysaccharide such as chitosan, carboxymethyl cellulose, hydroxypropyl cellulose, alginate, guar, starch, dextran, poly lactate, poly ascorbate, gelatin, xantham gum, glycans, welan guam, gellan gum, diutan gum, pullulan, and arabinoxylans and mixtures thereof) solutions decreases solution viscosity due—LHMC adsorption on liposomes through insertion of alkyl groups into the bilayer. The coating thickness can be modified through variations of polymer/lipid ratios. Visualization is done through cryo-TEM. The system transitions to a gel at higher concentrations of liposomes and polymer where the liposomes become nodes in a gel network. Cryo SEM confirms the presence of intact liposomes in the gel matrix.

Further embodiments of the present invention include the role of coating thickness in stabilizing liposomes against degradation in serum, the role of coating thickness on drug release kinetics, circulation kinetics of LHMC coated liposomes, the role of coating thickness in stabilizing liposomes against degradation in serum, the role of coating thickness on release kinetics, and circulation kinetics of LHMC coated liposomes.

The present invention includes a method of protecting liposomes, comprising providing the liposomes, and contacting the liposomes with a substance containing a hydrophobically modified polysaccharide.

Preferably, the hydrophobically modified polysaccharide comprises at least one from the group consisting of chitosan, carboxymethyl cellulose, hydroxypropyl cellulose, alginate, guar, starch, dextran, poly lactate, poly ascorbate, gelatin, xantham gum, glycans, welan guam, gellan gum, diutan gum, pullulan, and arabinoxylans and mixtures thereof.

Optionally, the liposomes can be spherical.

Optionally, the liposomes can be tubular.

Preferably, the polysaccharide comprises chitosan, carboxymethyl cellulose, alginate, and xantham gum.

Preferably, the polysaccharide can be chitosan.

Preferably, the hydrophobically modified polysaccharide can be a low molecular weight.

Preferably, the molecular weight can be about 50 k to 190 k Daltons.

Preferably, the molecular weight can be about 50 k Daltons.

Preferably, the mass ratio of hydrophobically modified polysaccharides to liposomes can be 0.1:1 to 1:1.

Preferably, the mass ratio of hydrophobically modified polysaccharides to liposomes can be 0.4:1.

Preferably, the hydrophobically modified polysaccharide creates a coating on the liposome.

Preferably, the coating thickness increases as the mass ratio increases.

Preferably, the liposomes can be used for drug delivery.

Optionally, the drug can be hydrophilic.

Optionally, the drug can be hydrophobic.

Optionally, the present invention further comprises a cyclodextrin in which a hydrophobic drug can be placed, and then put in the liposome.

Optionally, the hydrophobic drug can be inserted into the lipid bilayer of the liposome.

Preferably, the concentration of hydrophobically modified polysaccharide can be 0.4 wt % to 1.2 wt %.

Preferably, the concentration of hydrophobically modified polysaccharide can be 0.4 wt %.

Preferably, the thickness of the coating can be 20 nm.

The present invention includes liposomes coated with a hydrophobically modified polysaccharide.

The present invention includes tubular liposomes, produced by a method comprising:

a) providing a first material comprising a phospholipid which can be composed of a phosphate group and acyl chains;

b) providing a second material comprising a ceramide which can be composed of sphingosine and a fatty acid;

c) combining the first material and the second material to create a first mixture of the first and second materials;

d) providing a third material consisting of an organic chemical which can solubilize first material or/and the second material;

e) providing a fourth material consisting of an alcohol which can solubilize the first material or/and the second material;

f) combining the third material and the fourth material to create a second mixture of the third and fourth materials;

g) dissolving the first mixture in the second mixture to create a third mixture;

h) drying the third mixture until a dried lipid film is produced;

i) hydrating the dried lipid film with a fifth material consisting of a buffered solution to obtain a liposome solution;

j) sonicating the liposome solution; and

k) extruding the sonicated liposome solution to produce tubular liposomes in the extruded liposome solution.

Preferably, the tubular liposomes can have an aspect ratio (length to diameter) of at least 3.

Preferably, the tubular liposomes can be made by a method comprising:

a) providing a first material comprising a phospholipid which can be composed of a phosphate group and acyl chains;

b) providing a second material comprising a ceramide which can be composed of sphingosine and a fatty acid;

c) combining the first material and the second material to create a first mixture of the first and second materials;

d) providing a third material consisting of an organic chemical which can solubilize first material or/and the second material;

e) providing a fourth material consisting of an alcohol which can solubilize the first material or/and the second material;

f) combining the third material and the fourth material to create a second mixture of the third and fourth materials;

g) dissolving the first mixture in the second mixture to create a third mixture;

h) drying the third mixture until a dried lipid film is produced;

i) hydrating the dried lipid film with a fifth material consisting of a buffered solution to obtain a liposome solution;

j) sonicating the liposome solution; and

k) extruding the sonicated liposome solution to produce tubular liposomes in the extruded liposome solution.

Preferably, the tubular liposomes contain enzymes, magnetic particles, drugs or vaccines.

Preferably, the tubular liposomes can be 15 to 70 nm in diameter and 50 nm to 1 micron long.

Preferably, the first material can be selected from the group consisting of L-α-phosphatidylcholine, dipalmitoyl phosphatidylcholine, dimyristoyl phosphatidylcholine, and distearoyl phosphatidylcholine.

Preferably, the second material can be selected from the group consisting of ceramide VI and ceramide IIIA.

Preferably, the sonicated liposome solution can be repeatedly extruded through at least one multiple-nanometer pore size membrane.

Preferably, the tubular liposomes can be from the group consisting of undulating tubular liposomes and helical tubular liposomes and mixtures thereof.

Preferably, the tubular liposomes can be about 15 to 70 nm in diameter and 50 nm to 1 micron long.

Preferably, the tubular liposomes can be templated with silica.

Preferably, the functional groups can be chemically bound to the silica.

Preferably, the tubular liposomes can be consumed over a period of time in the body and the consumed liposomes allow a slow release of drugs or vaccines.

Preferably, the tubular liposomes can encompass a liquid.

Preferably, the tubular liposomes comprise:
a) a phospholipid which comprises a phosphate group and acyl chains; and
b) a ceramide which comprises sphingosine and a fatty acid.

Preferably, the tubular liposomes can be used deliver drugs or vaccines.

Preferably, the tubular liposomes can be made by a method comprising:

a) providing a first material from the group consisting of: L-α-phosphatidylcholine, dipalmitoyl phosphatidylcholine, dimyristoyl phosphatidylcholine, distearoyl phosphatidylcholine and other phospholipids which are composed of a phosphate group and acyl chains;

b) providing a second material from the group consisting of ceramide VI, ceramide IIIA, and other ceramides which are composed of sphingosine and a fatty acid;

c) combining the first material and the second material in a ratio by weight of about 80:20 to 25:75 to create a first mixture of the first and second materials;

d) providing a third material from the group consisting of chloroform, DMSO, THF, and other organic chemicals which can solubilize first material or/and the second material;

e) providing a fourth material from the group consisting of methanol, ethanol, butanol and other alcohols which can solubilize the first material or/and the second material;

f) combining the third material and the fourth material in a ratio by volume of about 1:10 to 10:1 to create a second mixture of the third and fourth materials;

g) dissolving the first mixture in the second mixture to create a third mixture;

h) drying the third mixture on a rotary evaporator (or under a dried inert gas stream) for about 1 or more hours until a dried lipid film is observed;

i) hydrating the dried lipid film with a fifth material from the group consisting of distilled water, phosphate buffered saline, and other buffered solutions to obtain about a 0.2-5.0% (weight/volume) liposome solution;

j) probe or bath sonicating the liposome solution; and

k) extruding the sonicated liposome solution through a series of 400 nm and 100 nm pore size polycarbonate membranes (or other membranes made of polymers of cellulose esters, or polyethersulfone) to produce tubular liposomes in the extruded liposome solution of about 15-70 nm in diameter and about 30- over 800 nm in length.

The present invention includes templated nanocontainers can be made by a method comprising:

a) providing the extruded liposome solution containing tubular liposomes;

b) diluting the extruded liposome solution about 2-10 fold with distilled water, PBS or any buffered solution to create a dilute solution;

c) adding to the dilute solution a silica precursor (such as TEOS, TMOS, aluminium silicate, or sodium silicate), a titania precursor (such as titanium isopropoxide, or titanium tetrachloride), or a calcium phosphate precursor (such as calcium chloride, potassium dihydrogen phosphate), to create a templating solution;

d) stirring the templating solution for about less than 1 day-21 days until templated nanocontainers are produced.

Preferably, the templated nanocontainers including drugs, enzymes, or other desired materials encapsulated therein, and made by a method comprising:

a) providing the templated nanocontainers;

b) adding of the drugs, enzymes, or other desired materials to the third material, the fourth material or the fifth material though dissolution, performing repeated freeze-thaw, or creating an active loading gradient for the drug, enzyme, or other desired materials.

Preferably, the tubular liposomes can be made with starting materials including a ceramide selected from the group consisting of ceramide VI and ceramide IIIA and mixtures thereof.

The present invention includes a composition used for drug delivery, comprising liposomes, and hydrophobically modified polysaccharides with alkyl groups, wherein the alkyl groups physically attach to and coat the liposomes.

### BRIEF DESCRIPTION OF THE DRAWINGS

For a further understanding of the nature, objects, and advantages of the present invention, reference should be had to the following detailed description, read in conjunction with the following drawings, wherein like reference numerals denote like elements and wherein:

FIG. 1 shows (a) photographs and (b) schematics that show the transition of liposome from a liquid to thick liquid and gel through the addition of HMC. (i) 1.0 wt % liposome, (ii) 1.0 wt % HMC+1.0 wt % DPPC&DMPG liposome, and (iii) HMC solution containing 1 wt % liposome and 1 wt % HMC results an elastic gel that is able to hold its own weight in the inverted vial. Dye used in samples: 0.0005 wt % methylene blue;

FIG. 2 shows cryo-SEM images of (a) native DPPC-DMPG liposomes, (b) HMC-coated liposomes at lower concentration of 0.4% HMC and (c) gel formed at relative higher concentration of 1% HMC;

FIG. 3 shows cryo-TEM of (a) native DPPC-DMPG liposomes and (b) HMC coated DPPC-DMPG liposomes at the mass ratio of HMC to lipids is 0.4:1, where a dark layer around liposomes was observed;

FIG. 4 shows cryo-TEM of liposomes (DPPC-DMPG) coated with various concentration of low molecular weight HMC: (a) bare liposomes, (b) 0.2% HMC coated, (c) 0.4% HMC coated, (d) 0.6% HMC coated;

FIG. 5 shows dynamic rheology of (a) HMC solution (0.6 wt % and 1.0 wt %) and (b) HMC-DPPC&DMPG liposomes (1 wt %) mixture;

FIG. 6 shows the apparent viscosity as a function of shear rate for different systems; and

FIG. 7 shows HMC coated liposomes after incubation in serum for 1 hour. HMC concentration: 0.4 wt % and liposome concentration: 1 wt %.

### DETAILED DESCRIPTION OF THE INVENTION

Detailed descriptions of one or more preferred embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

The present invention focuses on the application of hydrophobically modified chitosan (HMC) with liposomes. In addition to the use of HMC, the inventors believe that virtually any hydrophobically modified polysaccharide can be used. Some polysaccharides of potential use include chitosan, carboxymethyl cellulose, hydroxypropyl cellulose, alginate, guar, dextran, xanthan gum, welan guam, gellan gum, diutan gum, pullulan, arabinoxylans starch, poly lactate, poly ascorbate, gelatin, and glycans and mixtures thereof.

The present invention involves creating coatings on liposomes to increase stability within the body for drug delivery. The coating can be a hydrophobically modified polysaccharide, such as chitosan, carboxymethyl cellulose, hydroxypropyl cellulose, alginate, guar, starch, dextran, poly lactate, poly ascorbate, gelatin, xantham gum, glycans, welan guam, gellan gum, diutan gum, pullulan, and arabinoxylans and mixtures thereof.

The Effects of Hydrophobically Modified Chitosan (or Another Polysaccharide) on Liposomes:

We present results of work showing that chitosans that are hydrophobically modified with long alkyl groups have a protective influence on liposomes, allowing enhanced circulation times and sustained drug delivery. The alkyl groups partition into the lipid bilayers allowing the chitosan to form a coating that stabilizes the liposome. Such coated liposomes have extended circulation times and are protected from degradation by serum enzymes. We show the results of such coatings for spherical phospholipid liposomes and for a class of novel tubular liposomes obtained through the addition of sphingolipids (ceramides) to the phospholipid bilayer. Details of liposome structure, dynamics, and morphology are characterized through cryoelectron microscopy and high resolution NMR. As the concentration of hydrophobically modified chitosan is increased, the system transitions from a liquid to a gel where the liposomes act as nodes in a network structure. The transition is understood through detailed rheological characterization. Results are presented on drug release from such modified liposomes as correlated with liposome structure and system viscoelastic characteristics.

### Materials and Methods

### Materials.

DMPG (1,2-dimyristoyl-sn-glycero-3-(Phospho-rac-(1-glycerol)), DPPC (1, 2-dipalmitoyl-sn-glycero-3-Phosphocholine) and Mini-Extruder were from Avanti Polar Lipids, Alabaster, Ala. Polysaccharide, such as Chitosan used in this preparation, of low molecular weight (approximately 50K-190K Daltons, preferably 50 k Daltons) was obtained from Aldrich. The reported degree of deacetylation was between 75% and 85%. We have used 1% acetic acid to control the pH in chitosan solution. Dodecyl aldehyde, sodium cyanoborohydride (NaCNBH₃), sodium hydroxide, acetic acid, and ethanol were obtained from Sigma-Aldrich and were used as received without further treatment. Deionized (DI) water generated with a Barnstead E-pure purifier (IA) was used in all experiments.

### Preparation of Liposomes.

The liposomes used in this study were prepared by thin-film evaporation method as previously described. In detail, the phospholipids of DPPC and DMPG were mixed in the ratio 1:1 (w/w, 0.01 g-0.1 g, preferably 0.05 g each) and dissolved in 10 mL of chloroform and methanol mixture (2:1 v/v). The solution was evaporated by using a rotary evaporator (BUCHI, Switzerland) for 2.5 hours to form a dry lipid film. The lipid film was then hydrated for 1 hour with 5 mL of DI water at 50° C. and 125 rpm to obtain a 2% (w/v) liposome suspension. The liposome suspension was gently probe sonicated and subsequently extruded 11 times through a series of 400 nm and 100 nm pore size polycarbonate membranes (Whatman, Mobile, Ala.) at 55-65° C. to downsize the liposomes. The structures of DPPC and DMPG are provided in Scheme 1 (a) and (b).

### Synthesis of Hydrophobically Modified Chitosan (HMC).

HMC was derived by reaction the amine groups of chitosan with n-dodecyl aldehyde. All amine containing polysaccharides can be prepared through this route. The procedure used was identical to that described in the literature. Briefly, 1.0 g-10 g, preferably 4 g, of chitosan was firstly dissolved in 220 mL, of 1% (v/v) acetic acid, followed by the addition of 150 mL ethanol to allow the aldehyde used for the alkylation to be in a solvating medium. The pH was adjusted to 5.1 by the addition of sodium hydroxide and then the solution of dodecyl aldehyde in ethanol was added at 2.5% ratio to the chitosan monomole prior to an excess of sodium cyanoborohydride (3 moles per chitosan monomole). The mixture was stirred for 24 hours at room temperature and the final product was firstly precipitated with ethanol and sodium hydroxide solution, and then washed with ethanol and DI water three times.

### HMC Coated Liposomes.

To prepare HMC coated liposomes, an appropriate amount of the HMC polymer was firstly dissolved in 1% (v/w) acetate solution (pH=1) in order to prepare various HMC solutions that would results in concentration from 0.4 wt % up to 1.2 wt % (for example, with 1 ml of solution (1 g), we will need about 4 mg to 12 mg of HMC to produce 0.4 wt % to 1.2 wt %). In each case, an aliquot of the liposome dispersion was mixed with an equal volume of polymer solution, which was added dropwise to the liposomes, under continuous stirring. After this, the mixture was incubated for 30 min at room temperature. The resulted HMC coated liposome suspensions were stored in the refrigerator for further analysis.

### Analytical.

Cryo-transmission electron microscopy (TEM, JEOL 2011) was utilized to image the liposomes or HMC coated liposomes in their native state. In the process, a 10 μL drop of native liposome or HMC coated liposome suspension was placed on a Formvar coated copper TEM grid. The grid was blotted to form a thin film and rapidly vitrified in liquid ethane. The sample was then transferred under the protection of liquid nitrogen to a TEM equipped with a Gatan cold stage, and examined under acceleration voltage of 120 kV as the same as in a conventional TEM mode. The temperature of the sample grid was maintained at −175° C. during the course of imaging Cryo-SEM images were performed on a field-emission scanning electron microscope (SEM, Hitachi 4800). Briefly, the procedure involves rapid plunging of the sample into liquid nitrogen, followed by freeze-fracture using the flat edge of a cold knife (−130° C.) and then sublimation for 5 min at −95° C. to etch away surface water and expose internal features. The sample was then sputter coated with platinum at 10 mA for 88 s and imaged on the SEM at a voltage of 3 kV and at a working distance of 6 mm. The viscosity of HMC coated liposomes suspension is provided through rheological studies. The experiments were done at 25° C. on a TA Instruments AR 2000 rheometer using a concentric cylinder geometries set-up. Samples were placed between the parallel plates and sheared for 1 min at a large shear rate (˜80 s⁻¹) and zero field strength to ensure a uniform suspension distribution.

### Results and Discussion

### Phase Transition.

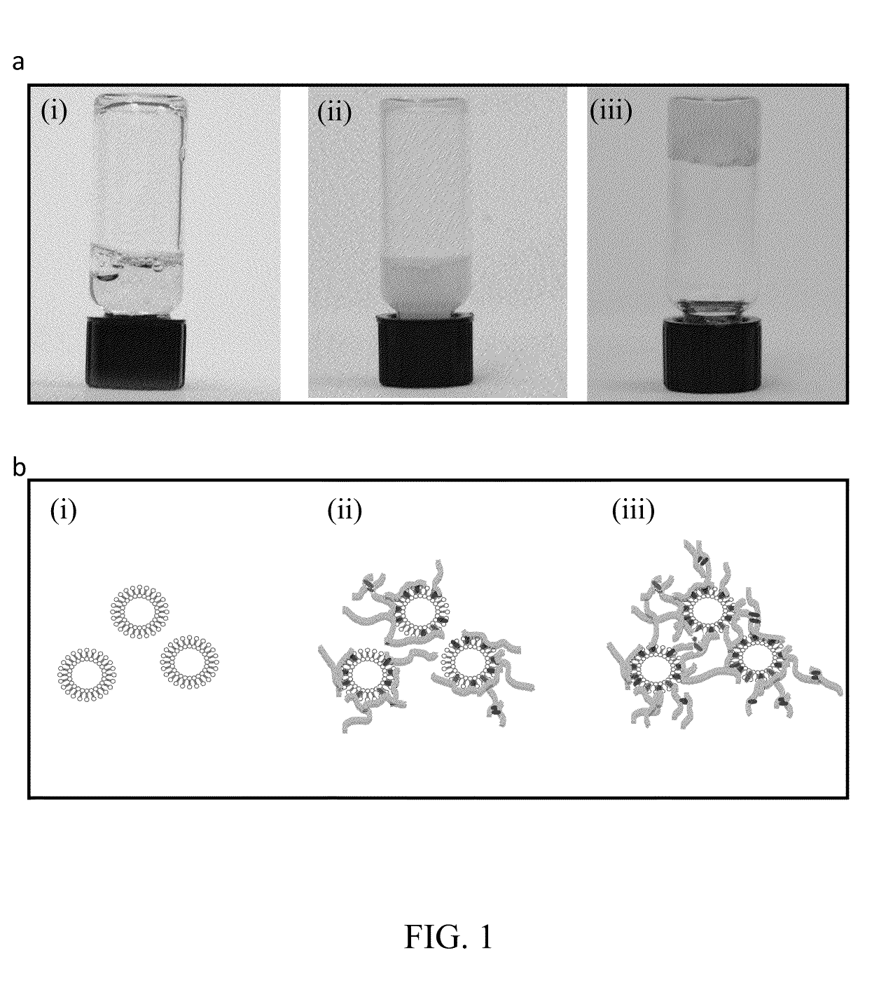

The effect of adding a hydrophobically modified polysaccharide, such as chitosan, carboxymethyl cellulose, hydroxypropyl cellulose, alginate, guar, starch, dextran, poly lactate, poly ascorbate, gelatin, xantham gum, glycans, welan guam, gellan gum, diutan gum, pullulan, and arabinoxylans and mixtures thereof, on the phase behavior of a 1 wt % solution of liposome is readily observed by vial tests. FIG. 1a shows a photograph of three samples: (i) a control of 1 wt % liposome, (ii) a moderately viscous solution containing 1 wt % liposome and 0.4 wt % HMC, and (iii) a gel containing 1 wt % liposome and 1 wt % HMC. We notice that the native liposome solution is a clear liquid, whereas a sample containing 0.4% HMC is an obscure fluid due to the presence of entangled wormlike polymers. Furthermore, upon adding 1% HMC, the sample is instantaneously transformed into a gel that is able to hold its own weight under vial inversion, which is identical to previous research results. All systems have been dyed with 0.0005% wt % methylene blue to distinguish them from background. In the coating process of HMC, the HMC solution was added into the liposome suspension dropwisely, it could be observed that flocculation emerged in the instillation process. FIG. 1b provides a schematic illustration of the proposed mechanism of HMC-induced phase transition. We assume that hydrophobes on the side chain of HMC tend to anchor within the liposome bilayers due to hydrophobic interaction (see FIG. 1(b)(i)). At the lower concentration of 0.4 wt % HMC, the polymer molecules are “dressed” on the layer of liposomes (see FIG. 1(b)(ii)). At sufficiently high concentrations, the HMC polymer can bridge liposomes, and the liposomes can serve as network junctions to yield an elastic gel (see FIG. 1(b)(iii)).

### Cryo Electron Microscopy Characterization

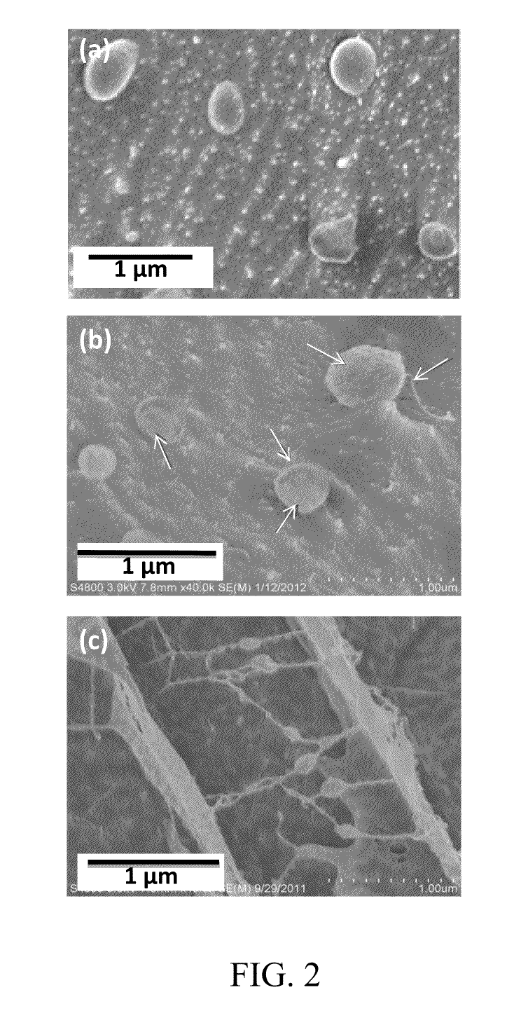

The morphology and microstructure of HMC-liposome assembly systems were analyzed through cryo scanning and transmission electron microscopy. FIG. 2 illustrates the cryo-SEM of HMC-liposome assembly systems showing a clear difference in morphology from the native liposomes. As shown in FIG. 2a, the native liposomes are a typical spherical structure, consistence with the literature. With the addition of a small amount of HMC at the concentration of 0.4%, these polymers are attached onto the layer of liposomes as suggested in FIG. 2b. Some of HMC chains protrude from the liposome surface as denoted by arrows. Our hypothesis is that hydrophobes on the side chain of HMC tend to insert within the liposome bilayers so that to form coatings on the liposomes. FIG. 2c presents cryo-SEM images of the gel, which is the result of adding relative large amount of HMC at the concentration of 1%. We observe cell-like structures with tendrils protruding from the cell walls, and liposomes dispersed throughout the gel phase. Although the addition of vesicles to polymer solutions results gel formation has been reported in the literature, the visualized characterization by electron microscopy of the interaction between vesicles and polymers within gels are seldom reported.

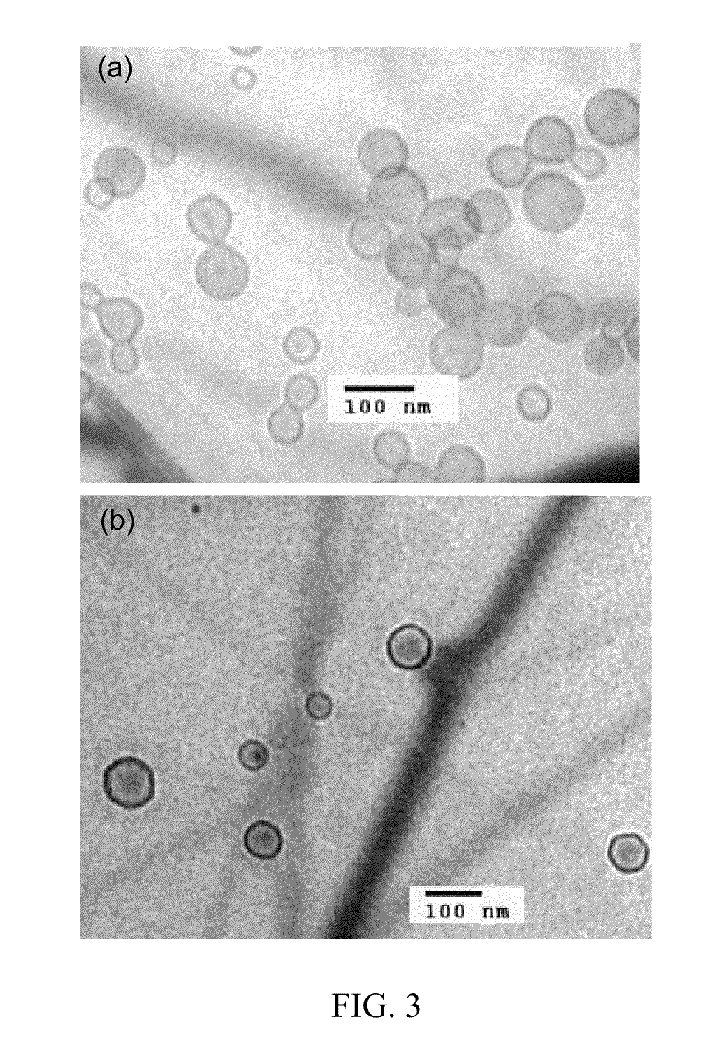

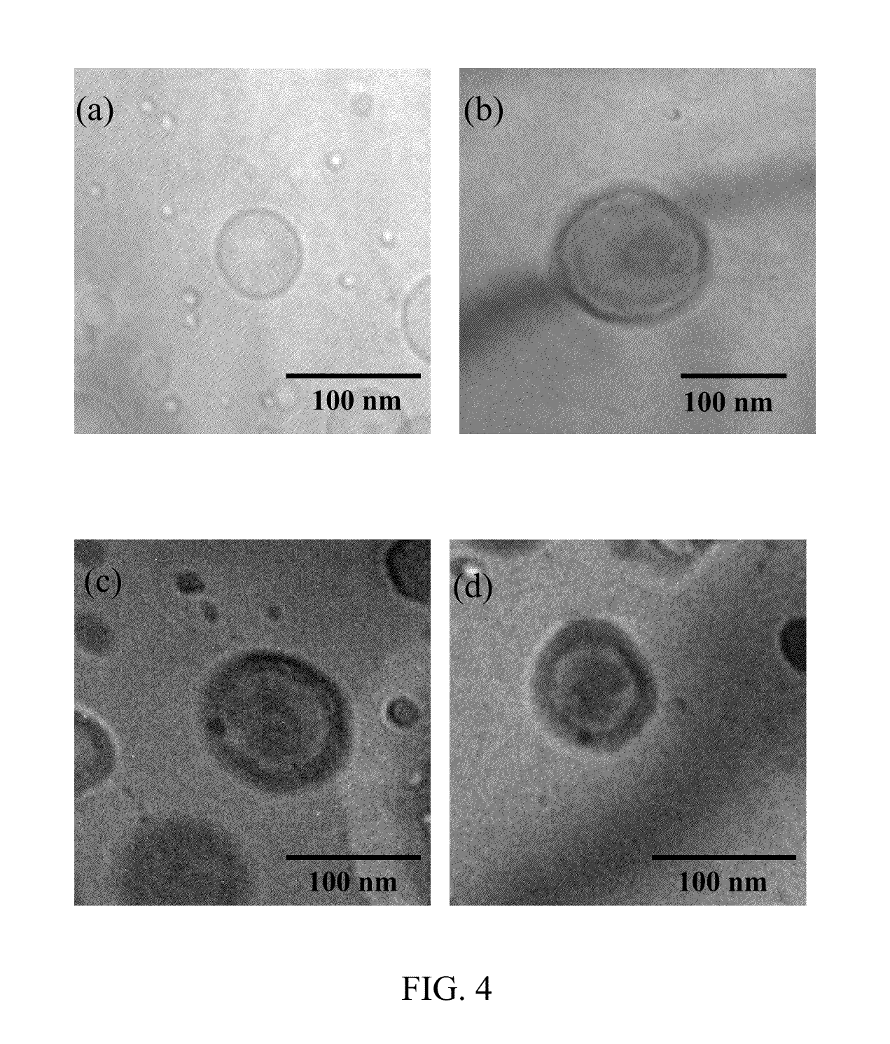

The HMC polymer creating coatings on the surface of liposomes are further investigated by cryo-TEM. As shown in FIG. 3, compared to native liposomes, HMC-coated liposomes show a thick shell of polymer molecules closely associated to the liposome surface. The influence of polymer concentration on morphology is further evaluated. FIG. 4 suggests the layer of liposomes becomes thicker with the increase of the amount of HMC, which further convinces that the polymer chains crouch on the surface of liposomes. As seen in FIG. 4, the coating layer of HMC has an approximate thickness of 20 nm. It is possible that the coating layer of HMC on each liposome is of varying thicknesses.

### Rheological Behavior

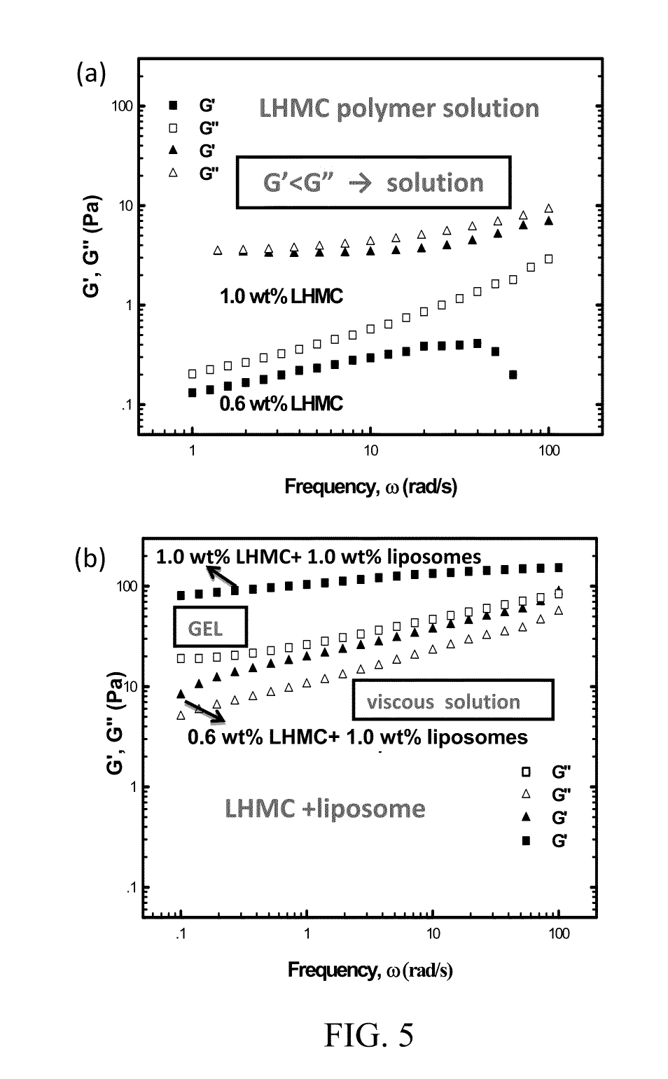

Qualitative evidence for phase transition of HMC-liposome systems is provided through rheological studies. FIG. 5 shows elastic modulus (G′) and viscous modulus (G″) vary as function of angular frequency for samples containing HMC, both without and with liposomes. FIG. 5a is a control experiment, which shows both solutions of HMC at the concentration of 0.6 wt % and 1.0 wt % exhibit a viscous response typical of weakly entangled polymer solutions. Here, the elastic modulus (G′) is lower than the viscous modulus (G″) over the range of frequencies, and both moduli show a strong frequency-dependence. Moreover, the fact that modulus of HMC at the concentration of 1.0 wt % is greater than ones at 0.6 wt % implies that hydrophobic crosslink between HMC chains can form easily at relative higher concentration.

FIG. 5b suggests the dynamic rheological behavior of HMC-liposomes systems. It is observed that the dynamic response of liposomes+1.0 wt % HMC satisfies the strict rheological definition of a gel, where G″ is greater than G′ with no dependence of the moduli on frequency. Furthermore the viscous modulus G″ is 10-fold higher for 1.0% HMC-liposome compared to bare HMC solution at the same concentration. This gel-like behavior is responsible to the ability of the sample to hold its weight under vial inversion. However, the system of 0.6% HMC-liposome still exhibits an elastic behavior where the value of G′ exceed G″ and both moduli being strong function of frequency. We assume this is because the lack of hydrophobic crosslink between HMC chains is not able to result in a gel at the lower amount of HMC with liposomes, which is in agreement with the cryo-SEM and cryo-TEM images.

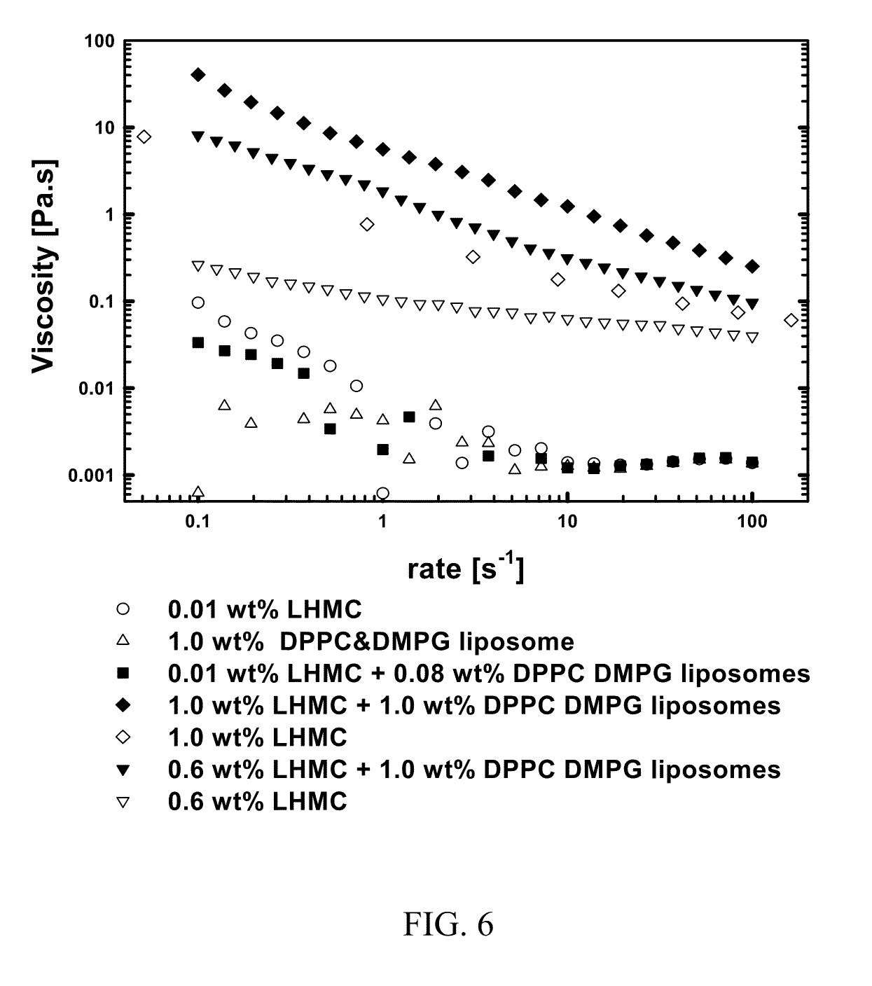

FIG. 6 shows the apparent viscosity as a function of shear rate for various systems including HMC solution, HMC-coated liposomes and HMC-liposomes gel. Taking the case of the native low concentration HMC (0.01 wt %) first, the addition of liposomes (0.08 wt %) decreases the viscosity. This suggests that polymer hydrophobes prefer to insert into the liposomes bilayers and thus limit the entanglement of the polymer chains, confirming our previous hypothesis that HMC polymer is able to coat liposomes due to hydrophobic interactions between hydrophobes on the side chain of HMC and liposome bilayers. It also notes that there is an entirely different phenomenon for high concentrations of HMC, where the HMC-liposome systems have a greater viscosity than native polymer solutions. We assume that HMC polymers coated on the liposomes exists but with a small effect on viscosity properties, because it cannot compete with the fact that the liposomes may act as connection nodes for HMC chains thereby increasing HMC solution viscosity. This is further confirmed by the fact that the viscosity is increased with the increase of HMC.

### Liposome Stability

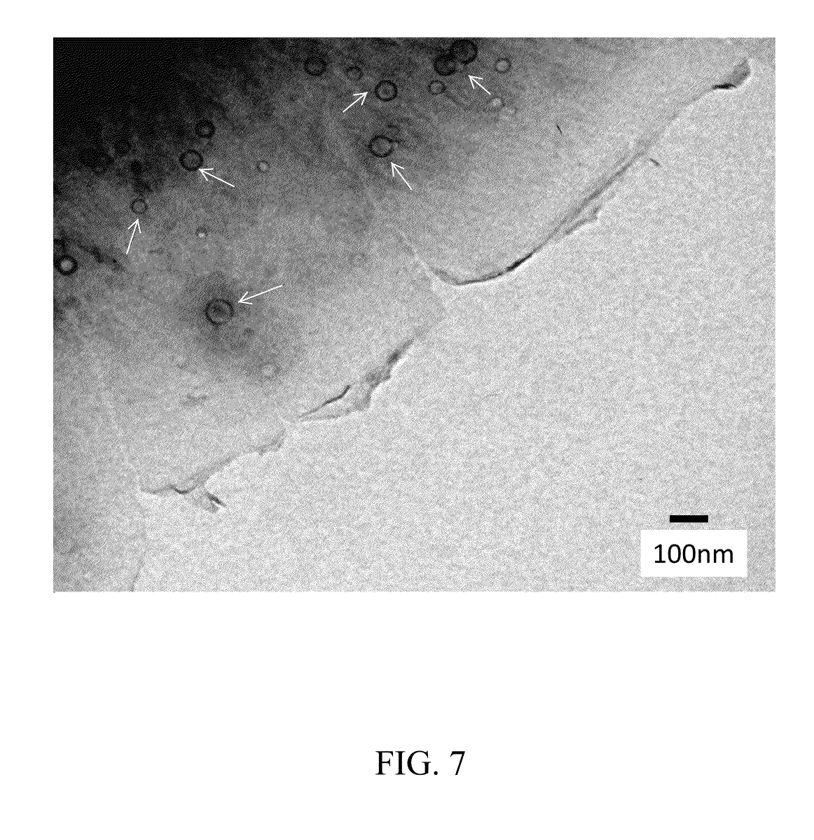

Liposome stability is defined as the ability to retain liposome structural integrity and prevent leakage of entrapped contents. We noted that uncoated liposomes are not visualized by cryo-TEM after incubation in 10% fetal bovine serum solution for 1 hour. This means native liposomes are not stable and self-closed phospholipid bilayers are destroyed in serum solution. However, under the same condition, HMC-coated liposomes can be clearly observed as shown in FIG. 7. The similarity between the images of HMC-coated liposomes before and after incubation convinced the polymer as a coating enhanced the stability of liposomes, adding evidence to the hypothesis that HMC-coated liposomes with an increased carrier potential for application in vivo.

### Use of Liposomes

The coated liposomes of the present invention can be primarily used as pharmaceutical or nutraceutical drug carriers to deliver compounds in the human body. Hydrophilic drugs can often be placed in liposomes and coated with a hydrophobically modified polysaccharide, such as HMC, using the methods disclosed herein. When the drugs are hydrophobic, it is sometimes advantageous to first put the drugs in cyclodextrin, then put the cyclodextrin in the liposome, then coat with HMC. Alternatively, hydrophobic drugs can also be inserted into the lipid bilayer of the liposomes without the use of cyclodextrins.

Cyclodextrins can also be added to the liposome-HMC solution to assist with removing of the HMC coating from the liposome. This is useful in making the liposome a quick-release vessel.

Cyclodextrins (CDs) and liposomes have been used in recent years as drug delivery vehicles, improving the bioavailability and therapeutic efficacy of many poorly water-soluble drugs. The amount of lipophilic drug incorporated into the conventional liposome bi-layer is often limited in terms of drug to lipid ratio. The combined approach of using CDs and liposomes has established a novel system of DCL (Drug-in-Cyclodextrin-in-Liposome) preparation for the delivery of water-insoluble compounds such as for example curcumin. Cyclodextrin complexation improved drug solubilization and allowed an improvement of its entrapment into the aqueous liposomal phase.

Liposomes are colloidal entities in aqueous solution that consist of one or more lipid bilayers enclosing an inner aqueous phase. They are typically spherical with sizes ranging from 20 nm (nanometers) to 10 um (microns). In biology, this specifically refers to a membrane composed of a phospholipid and cholesterol bilayer. Liposomes can be composed of naturally-derived phospholipids with mixed lipid chains (like egg phosphatidylethanolamine), or of pure surfactant components like DOPE (dioleoylphosphatidylethanolamine). Liposomes, usually but not by definition, contain a core of aqueous solution; lipid spheres that contain no aqueous material are called micelles; however, reverse micelles can be made to encompass an aqueous environment.

Liposomes are used for drug delivery due to their unique properties. A liposome encapsulates a region of aqueous solution inside a hydrophobic membrane; dissolved hydrophilic solutes cannot readily pass through the lipids. Hydrophobic chemicals can be dissolved into the membrane, and in this way liposome can carry both hydrophobic molecules and hydrophilic molecules. The ability to encapsulate hydrophilic compounds such as proteins in the aqueous core of the liposomes and simultaneously incorporate lipophilic drugs in the hydrophobic lipid bilayer specifically renders liposomes as suitable vehicles for drug delivery. To deliver the molecules to sites of action, the lipid bilayer can fuse with other bilayers such as the cell membrane, thus delivering the liposome contents. By making liposomes in a solution of DNA or drugs (which would normally be unable to diffuse through the membrane) they can be (indiscriminately) delivered past the lipid bilayer. Liposomes can also be designed to deliver drugs in other ways. Liposomes that contain low (or high) pH can be constructed such that dissolved aqueous drugs will be charged in solution. As the pH naturally neutralizes within the liposome (protons can pass through some membranes), the drug will also be neutralized, allowing it to freely pass through a membrane. These liposomes work to deliver drug by diffusion rather than by direct cell fusion. Another strategy for liposome drug delivery is to target endocytosis events. Liposomes can be made in a particular size range that makes them viable targets for natural macrophage phagocytosis. These liposomes may be digested while in the macrophage's phagosome, thus releasing its drug. Liposomes can also be decorated with opsonins and ligands to activate endocytosis in other cell types.

Since the coating is positively charged/cationic, it is possible to attach negatively charged substances, such as DNA, and anionic particles, such as magnetic iron oxides, to the liposome for delivery. This allows the liposome with coated HMC to serve as a vehicle for gene delivery to the cell. It is possible to deliver multiple agents to the cell, with drugs in the interior of the liposome and DNA on the exterior attached to the HMC.

The coating also makes the liposomes more stable for transportation.

### REFERENCES

### All of which are Incorporated Herein by Reference

Lee, J. H.; Gustin, J. P.; Chen, T. H.; Payne, G. F.; Raghavan, S. R., Vesicle-biopolymer gels: Networks of surfactant vesicles connected by associating biopolymers. Langmuir 2005, 21, (1), 26-33.
Zhu, C.; Lee, J. H.; Raghavan, S. R.; Payne, G. F., Bioinspired vesicle restraint and mobilization using a biopolymer scaffold. Langmuir 2006, 22, (7), 2951-2955.
Dhule, S. S.; Penfornis, P.; Frazier, T.; Walker, R.; Feldman, J.; Tan, G.; He, J.; Alb, A.; John, V.; Pochampally, R., Curcumin-loaded γ-cyclodextrin liposomal nanoparticles as delivery vehicles for osteosarcoma. Nanomedicine: Nanotechnology, Biology and Medicine 2012, 8, (4), 440-451.
Yamada, M.; H. Harashima; Kiwada, H., Kinetic analysis of the interaction between liposomes and the complement system in rat serum: Re-evaluation of size-dependency. Biol. Pharm. Bull 1998, 21, (9), 964-968.
C. M. Aberg, T. H. Chen, A. Olumide, S. R. Raghavan and G. F. Payne, J. Agric. Food Chem., 2004, 52, 788-793 (DOI:10.1021/jf034626v).
J. Yang, F. Tian, Z. Wang, Q. Wang, Y. Zeng and S. Chen, Journal of Biomedical Materials Research Part B: Applied Biomaterials, 2008, 84B, 131-137 (DOI:10.1002/jbm.b.30853).
Q. Z. Wang, X. G. Chen, Z. X. Li, S. Wang, C. S. Liu, X. H. Meng, C. G. Liu, Y. H. Lv and L. J. Yu, Journal of Materials Science: Materials in Medicine, 2008, 19, 1371-1377 (DOI:10.1007/s10856-007-3243-y).
B. C. Dash, G. Réthoré, M. Monaghan, K. Fitzgerald, W. Gallagher and A. Pandit, Biomaterials, 2010, 31, 8188-8197 (DOI:DOI: 10.1016/j.biomaterials.2010.07.067).
El-Mekawy, S. Hudson, A. El-Baz, H. Hamza and K. El-Halafawy, J. App. Poly. Sci., 2010, 116, 3489-3496 (DOI:10.1002/app.31910).

### ACRONYMS

CD cyclodextrin

### DCL Drug-in-Cyclodextrin-in-Liposome

DI deionized

### DMPG Dimyristoyl-sn-Glycero-3-PhosphoGlycerol

DOPE dioleoylphosphatidylethanolamine

### DPPC Dipalmitoylphosphatidylcholine

HM hydrophobically modified
hm-chitosan/hmC/HMC hydrophobically modified chitosan
IA Barnstead E-pure purifier
LHMC low molecular weight hydrophobically modified chitosan
NMR nuclear magnetic resonance
PEG Polyethylene glycol
RES reticuloendothelial system
SEM scanning electron microscope
TEM transmission electron microscopy

All measurements disclosed herein are at standard temperature and pressure, at sea level on Earth, unless indicated otherwise. All materials used or intended to be used in a human being are biocompatible, unless indicated otherwise.

The foregoing embodiments are presented by way of example only; the scope of the present invention is to be limited only by the following claims.

1. A method of protecting liposomes, comprising:
a) providing the liposomes; and
b) contacting and coating the liposomes with a substance containing a hydrophobically modified polysaccharide.

2. The method of claim 1, wherein the hydrophobically modified polysaccharide comprises at least one from the group consisting of chitosan, carboxymethyl cellulose, hydroxypropyl cellulose, alginate, guar, starch, dextran, poly lactate, poly ascorbate, gelatin, xantham gum, glycans, welan guam, gellan gum, diutan gum, pullulan, and arabinoxylans and mixtures thereof.

3. (canceled)

4. The method of claim 1, wherein the polysaccharide comprises chitosan, carboxymethyl cellulose, alginate, and xantham gum.

5-6. (canceled)

7. The method of claim 1, wherein the hydrophobically modified polysaccharide has a molecular weight that is 50 k to 190 k Daltons.

8. (canceled)

9. The method of claim 1, wherein the mass ratio of hydrophobically modified polysaccharides to liposomes is 0.1:1 to 1:1.

10-11. (canceled)

12. The method of claim 9, wherein the coating thickness increases as the mass ratio increases.

13. (canceled)

14. The method of claim 1, wherein the liposomes are used for delivery of at least one material from the group consisting of drugs, proteins and DNA, and wherein the material is hydrophophilic or hydrophobic.

15-16. (canceled)

17. The method of claim 14, further comprising a cyclodextrin in which the material is placed, and then put in the liposome interior or bilayer.

18-19. (canceled)

20. The method of claim 1, wherein the concentration of hydrophobically modified polysaccharide is 0.4 wt % to 1.2 wt %.

21. (canceled)

22. The method of claim 1, wherein the thickness of the coating is 20 nm.

23-62. (canceled)

63. A composition used for delivery of at least one material from the group consisting of drugs, proteins, and DNA, comprising:
a) liposomes; and
b) hydrophobically modified polysaccharides with alkyl groups, wherein the alkyl groups physically attach to and coat the liposomes.

64. The composition of claim 63, wherein the polysaccharides comprise at least one from the group consisting of chitosan, carboxymethyl cellulose, hydroxypropyl cellulose, alginate, guar, starch, dextran, poly lactate, poly ascorbate, gelatin, xantham gum, glycans, welan guam, gellan gum, diutan gum, pullulan, and arabinoxylans and mixtures thereof.

65. The composition of claim 63, wherein the polysaccharide comprise chitosan, carboxymethyl cellulose, alginate, and xantham gum.

66-67. (canceled)

68. The composition of claim 63, wherein the hydrophobically modified polysaccharide has a molecular weight that is 50 k to 190 k Daltons.

69. (canceled)

70. The composition of claim 63, wherein the mass ratio of hydrophobically modified polysaccharides to liposomes is 0.1:1 to 1:1.

71-74. (canceled)

75. The composition of claim 63, wherein the material is hydrophilic.

76. The composition of claim 63, wherein the material is hydrophobic.

77. The composition of claim 63, further comprising a cyclodextrin in which the material is placed, and then put in the liposome interior or bilayer.

78. (canceled)

79. The composition of claim 63, wherein the concentration of hydrophobically modified polysaccharide is 0.4 wt % to 1.2 wt %.

80. (canceled)

81. The composition of claim 63, wherein the thickness of the coating is 20 nm.

82. (canceled)
